(19) United States
(12) Patent Application Publication
(10) Pub. No.: US 2022/0233880 A1
(43) Pub. Date: Jul. 28, 2022

(54) INFRARED RAY EQUIPMENT FOR ROBOTIC REHABILITATION ON A TREADMILL, HAVING FLEXIBLE PELVIC ATTACHMENT

(71) Applicant: PANDHORA S.R.L., Napoli (IT)

(72) Inventors: Stefano TRONCONE, Napoli (IT); Alexander TRONCONE, Napoli (IT); Chrystel TRONCONE, Napoli (IT)

(73) Assignee: PANDHORA S.R.L., Napoli (IT)

(21) Appl. No.: 17/617,216

(22) PCT Filed: Jun. 4, 2020
(86) PCT No.: PCT/IB2020/055260

§ 371 Date: Dec. 7, 2021

(30) Foreign Application Priority Data
Jun. 11, 2019 (IT) 2019000001812

(51) Int. Cl. A61N 5/06; A61H 1/02; A63B 22/02

(52) U.S. Cl. CPC A61N 5/0625; A61H 1/0262; A63B 2022/0094; A61H 1/0274; A63B 22/02

(57) ABSTRACT

Rehabilitation device, comprising a motorized tapis roulant, a harness configured to support the weight of the patient; a plurality of infrared light sources, each one directed to light the muscles of a zone of the lower limbs of the patient; a motorized exoskeleton for lower limbs, configured such that it is fastened to the patient femur, tibia and foot and such that it guides his movements while walking in a combined manner with the movement of said tapis roulant; a user interface, arranged in a position visible for the user; a data processing and control unit, configured to control said motorized exoskeleton and said light sources, characterized in that said processing unit is configured to manage an intensity adjusting cycle of said light sources in a differentiated manner for each one of said sources, as a function of the movement cycle imposed by said exoskeleton to the lower limbs.

### BACKGROUND OF THE INVENTION

### 1. Field of the Invention

The present invention relates to the technical field of robotic rehabilitation apparatuses. In particular, the present invention relates to a robotic rehabilitation infrared apparatus on tapis roulant, with flexible pelvic attachment.

### 2. Brief Description of the Prior Art

The present invention aims at developing a robotic automatized system which allows a user to carry out robotic rehabilitation combined with physical and proprioceptive stimulations so to speed up and maximize the rehabilitation process.

Robotic rehabilitation allows to come back to an almost normal life even after traumas and invalidating pathologies. The use of robot physiotherapists has cleared a new way for the treatment of permanent and temporary disability. They are in fact exoskeletons and other robotic systems for limbs rehabilitation: wearable robots able to give back at least a partial mobility to people who have lost the use of their legs and hands.

The rehabilitation systems available today at the state of the art provide mechanical stimulations by compelling the user to carry out forced movements only combined with purely visive stimulations, which are apt to provide a proprioceptive stimulation in order to speed up the rehabilitation recovery process during the movement.

Anyway, rehabilitation techniques exist, based on the use of infrared radiation.

Infrared rays have a wavelength between 7600 and 150000 angstrom, but radiations about 40000 angstrom are commonly used in clinical practice. Discovered in 1800 by an English astronomer, infrared rays are not visible by naked eye. Inside the light spectrum, the infrared radiation range is immediately near the red range of visible light. So, they are low frequency rays and are able to emit heat. This feature is just the one that favored its application in medical and esthetic field.

The infrared radiation main effect is a thermal action, for this reason such therapy form is used to prepare the patient to massage or kinesitherapy. In fact, mild heat has a sedative and relaxing effect. The local application is carried out for 20-30 minutes with a lamp positioned at 50-60 cm from the zone to be treated considering that this has never to be urent.

The zone to be treated has to be naked and the radiations penetrate up to 0.5-10 mm under the skin.

Infrared rays are rapidly absorbed by the surface layers of the skin, so their penetration capacity is limited. The penetration degree is inversely proportional to wavelength.

And this is just the limit of the therapy: the penetration capacity. The main biological effect of infrared radiation is the thermal one. The infrared rays produce heat when they are absorbed by surrounding tissues.

The heat produced is partially dispersed in the environment and partially transmitted to profound tissues by conduction and by means of circulating liquids

As secondary effects, the temperature increase causes the increase of tissue metabolism, vasodilation of capillaries and arterioles and muscle relaxation.

Surface tissues are mainly affected by these effects.

The therapeutic effects of infrared radiation are mainly due to temperature increase in irradiated tissues and are represented by muscle relaxation, analgesia, trophic effect.

The antalgic effect of infrared radiation is attributed to the elimination of algogenic substances from pathologic tissues and to relaxation of contracted muscles.

The increase in blood flow, which follows the vasodilation, makes tissues receive a greater quantity of nutritive substances, oxygen, white blood cells and antibodies and simplifies the elimination of catabolites from tissues.

These biological modifications improve trophism of tissues, facilitate reparation of tissue damages and speed up the solution of chronic inflammatory infiltrates.

The heat, these rays transmit, is absorbed easily by the body, since it penetrates only the surface of skin and tissues, and causes a beneficial vasodilation besides stimulations at a nervous and muscular level. The treatment with infrared radiation is indicated in particular to treat specific disorders associated to circulatory and skeletal systems and to muscles.

Infrared rays are used in rehabilitation to contrast contractures and muscular pain and to prepare the muscle to massage and physiotherapy treatments.

This because, when they are directed on the zone to be treated, they heat in depth, thus determining a dilation of the blood vessels present in the treated zone with an increase in local blood circulation, and, as a consequence, a greater oxygenation of tissues, which regenerate.

Moreover, it is added a stimulating effect on nerve endings, which reduces pain and relaxes muscles.

So, they are indicated for the treatment of cervical pain, for cervical and lumbar arthrosis, for muscular contractures and for the treatment of bedsores.

Today, at the state of the art, many apparatuses exist which allow to carry out robotic rehabilitation and use tapis roulant to walk while the user is supported by a weight discharge system and/or where walking on tapis roulant is supported also by a leg orthosis or brace and the weight of such devices can be lightened by other weight lightening elements as a parallelogram support frame.

EP1137378 describes an automatic machine, which is used in the tapis roulant therapy (walking therapy) which can be used for paraparetic and hemiparetic patients and which guides the legs automatically on the tapis roulant. This machine is made up of a guided and controlled orthotic device, which guides the legs in a physiological model of movement, a tapis roulant and a discharge mechanism. The orthotic device knee and hip joints are provided each with a disk.

Such orthotic device is stabilized on a tapis roulant with stabilization means such that the patient has not to keep his balance. The orthotic device can be height-adjusted and can be easily adapted to different patients.

Further developments of the mechanism relate to a device to adjust the height and the discharge force acting on a weight, as it is described in EP1586291, which shows two different cable length adjusting means. The one is provided to adjust the cable length to define the height of the suspended weight. The other one is provided to adjust the cable length to define the discharge force acting of the suspended weight. Another mechanical solution to adjust the discharge force can be found in EP 1 908 442. WO 2010/105773, KR 2013 0038448, WO 2012/178171, US 2007/270723 and US 2007/004567 describe a walking training device.

Another example is described in the application PCT/EP2014/063053 which describes an apparatus for automatized training of a user on tapis roulant which comprises: a frame configured to allow the user to walk, a pelvic attachment to support the weight or the position of the user and having attachment elements configured to be connected to the user, wherein the pelvic attachment comprises a displacement unit to allow and support the movement of the user pelvis held by the transversal fixing elements, by rotating around or transversely to an axis perpendicular to a deambulation direction.

In this Patent PCT/EP2014/063053 it is added a pelvic displacement unit which moves the user pelvis during deambulation.

But this solution does not consider the fact that the movement induced by the exoskeleton system to the lower limbs is imposed in a forced manner to the user. The addition of a displacement unit which moves forcedly also the pelvis, causes the pelvis muscles and glutei not to carry out any free movement, and this can cause detachments of the pelvis muscles and glutei during the forced movement. In fact, pelvis movement needs not to be induced, but it would be useful it to be freely carried out during the forced movement of the legs on the tapis roulant. In fact, this would allow the muscles not to be detached from bones, but on the contrary, this could lead to their natural compression on bones, thus making the movement more natural.

Disadvantageously, the systems known at the state of the art have no physical system acting in a combined manner on muscles during movement.

### SUMMARY OF THE INVENTION

Advantageously, the apparatus on tapis roulant for robotic rehabilitation proposed by this invention is based on an infrared system combined with the movement induced to the user.

Moreover, the user pelvis is fastened by means of a flexible pelvic attachment which does not allow the muscle to be detached from bones, but it accompanies it freely during the forced movement of the legs.

Aim of the present invention is to facilitate the robotic rehabilitation by using infrared radiation. Yet another aim of the present invention is to combine the infrared radiation emission with the movement induced to the user.

Yet another aim of the present invention is to provide a physical stimulation during the movement induced by the robotic rehabilitation.

Yet another aim of the present invention is to provide a simultaneous physical stimulation, by heating the various muscles in a differentiated way.

Yet another aim of the present invention is to provide a simultaneous proprioceptive stimulation visible to the user during the differentiated light sources switching on, on the different muscular bands.

On the basis of further aspects, aim of the present invention is to provide an apparatus which by means of electro-stimulation combined with induced movement, stimulates muscles during robotic rehabilitation.

### BRIEF DESCRIPTION OF THE DRAWINGS

FIG. 1 shows an overall view of a preferential embodiment of the device according to the invention.

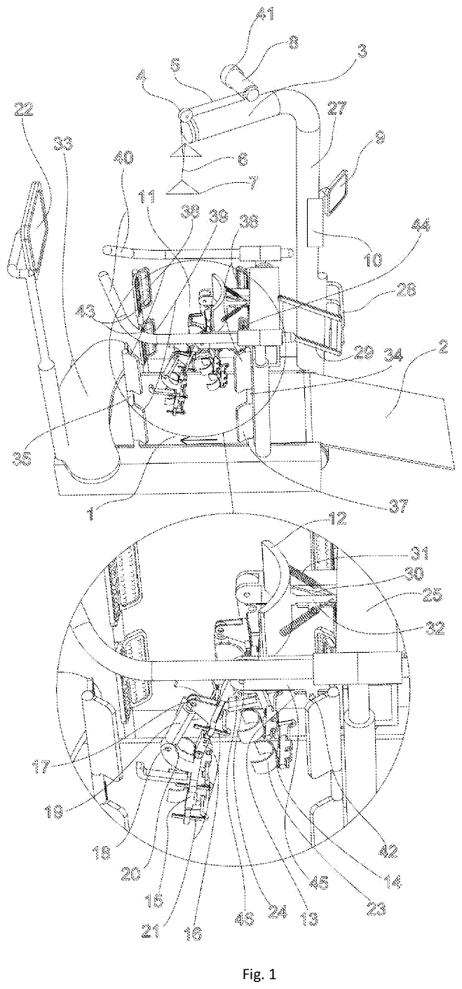

FIG. 2 shows a side view of the device according to the invention with the user using it.

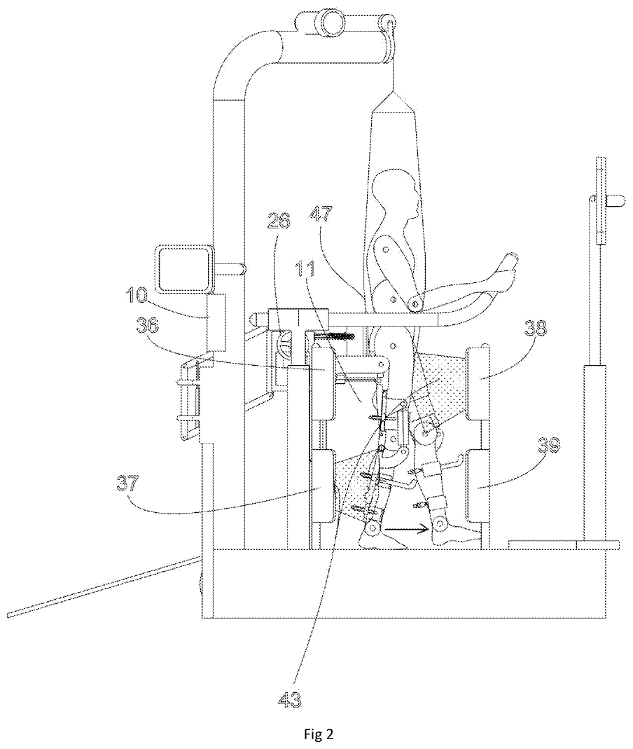

### DETAILED DESCRIPTION OF THE PREFERRED EMBODIMENTS

Such problem is solved with the infrared apparatus device for robotic rehabilitation on tapis roulant, having flexible pelvic attachment according to claim 1.

Preferred features of the invention are defined in the dependent claims.

The present invention realizes the prefixed aims since it is an infrared apparatus for robotic rehabilitation, having a flexible pelvic attachment, which comprises:

a tapis roulant (1), provided in its rear part with an access footboard (2) at tapis roulant level and in the front part with a banana shaped pillow

(33) arranged at tapis roulant level, wherein on the side of said tapis roulant (1) a pillar (27) is integral, which, on its upper end has a flag arm (3) extending at a height upper than a person's one up to the center of said tapis roulant (1);

return pulley (4), hinged on the end of said flag arm (3), on which a cable (5) slides which is connected in turn to a motorized winch (8), whose position is determined by an encoder (41) mounted on the rotation axis of said motorized winch (8); an equalizer (6), connected centrally to the other end of the cable (5), on whose ends (7) a harness (47) is connected;

a rear couple of vertical telescopic supports

(34), the lower end being fastened integrally to each side of the tapis roulant (1);

a first couple of rear, upper panels (36) fastened on the top to the rear couple of vertical telescopic supports (34), provided with infrared light sources (43) directed so to be incident on the rear on the leg muscles at the femoral zone; a second couple of rear, lower panels (37), fastened on the bottom to the rear couple of vertical telescopic supports (34), provided with infrared light sources (43) directed so to be incident on the rear on the leg muscles at the ankle zone;

a front couple of vertical telescopic supports (35), the lower end being fastened integrally to each side of the tapis roulant (1);

a third couple of front, upper panels (38) fastened on the top to the front couple of vertical telescopic supports (35), provided with infrared light sources (43) directed so to be incident frontally on the leg muscles at the femoral zone; a fourth couple of front, lower panels (39) fastened on the bottom to the front couple pf vertical telescopic supports (35), provided with infrared light sources (43) directed so to be incident frontally on the leg muscles at the ankle zone;

a couple of supports for arms (40) which can be height-adjusted and which can be drawn close to each other, fastened to each side of the tapis roulant (1);

an arm (28), hinged at mid height on the pillar (27), on whose end a parallelogram kinematism structure (29) is fastened, which can be lifted with the support of a gas spring provided in the kinematism, on whose vertical side opposite to the fastened one a transversal horizontal arm (25) is fastened, from which a little arm (32) begins;

a horizontal pelvic support (12), hinged centrally on the end of the little arm (32), maintained in its operational configuration, in transversal position to the tapis roulant (1) by means of variable rigidity springs (31), hinged with an end to each side of the horizontal pelvic support (12) and with the other end to a point of the little arm (32);

an adjustable dampening hydraulic dampener (30), hinged with an end to a side of the horizontal pelvic support (12) and with the other end to a point of the little arm (2);

fifth couple of upper, central panels (44) fastened by means of a connection link on the transversal horizontal arm (25), provided with infrared light sources (43) directed so to be incident on the rear on the glutei zone;

a motorized exoskeleton for lower limbs (11) made up, for each side, of a horizontal upper link (45) fastened to the transversal horizontal arm (25), on said horizontal upper link (45) an upper orthosis connection link (16) being hinged, actuated by an upper linear actuator (13) provided with position and force reader (24), said upper linear actuator (13) being fastened on an end to the horizontal upper link (45) by means of a first upper hinge (42) integral to a little arm and on the opposite end being fastened to the upper orthosis connection link (16) by means of a first lower hinge (46) integral to a little arm, moreover the upper orthosis connection link (16) is hinged on the opposite end to a lower orthosis connection link (15), actuated by a lower linear actuator (19) provided with position and force reader (20), said lower linear actuator (19) being fastened on an end to the upper orthosis connection link (16) by means of a second upper hinge (17) integral to a little arm and on the opposite end being fastened to the lower orthosis connection link (15) by means of a second lower hinge (18) integral to a little arm, moreover said horizontal upper links (45) are provided with drawing close kinematism between two leg orthoses by means of worm screw and actuation handwheel (26);

connection little arms (21) which slide by means of guides which can be blocked on the upper orthosis connection link (16) and on the lower orthosis connection link (15), at the end of each connection little arm (21), by means of hinges (14), being hinged band attachments (23) for leg;

a user interface monitor (22) positioned in front of the tapis roulant (1), in visible position for the user who uses said tapis roulant (1);

a touch screen programming unit (9), arranged on a side of the tapis roulant (1), from said touch screen programming unit (9) being programmed the movement of the tapis roulant (1), the intensity of the infrared light sources (43) and the automatized movement of the motorized exoskeleton for lower limbs (11);

a data processing and control unit (10), which manages the switching on frequency, and the intensity of the infrared light sources (43) in a differentiated manner between the rear, upper panels (36), the rear, lower panels (37), the rear upper panels (38), the front, lower panels (39) and the central upper panels (44) combining it with the automatized movement of the motorized exoskeleton for lower limbs (11) and with the movement of the tapis roulant (1).

In this way, the action of the infrared radiation combined with the movement of the exoskeleton provides a physical action, since it heats the muscle interested by the movement, at the same time the action becomes also a proprioceptive stimulation for the user since the switching on combined with the single movement results visible by the user who, after perceiving it, will combine it sensorially to the movement.

Moreover, on the pillar (27) it is possible to connect also an exoskeleton of upper limbs controlled by the data processing and control unit (10), provided with infrared light sources panels which irradiate in a differentiated manner the arms combining the irradiation in a differentiated manner with the movement of said exoskeleton of upper limbs. The banana shaped pillow (33) allows a therapist to help the user to hook the lower limbs orthoses to the legs, while being able to sit at the right, the left and at the center of the tapis roulant (1) at the tapis roulant level. The light sources can also have a wavelength different from the infrared one.

On the lower and possibly upper limbs orthoses electrodes can be arranged, in contact to legs and arms muscles, which electrodes, controlled by the data processing and control unit (10) stimulate muscles by means of the impulses sent in combined manner with the movement induced by the lower and upper limbs orthoses.

Moreover, in order not to overheat skin and to maximize the irradiation, the light sources can emit light in pulsed form.

In the following, it is specified a preferred embodiment of the combination of the stimulation by means of infrared light sources with the action of the motorized exoskeleton.

As yet described, the device comprises:

a tapis roulant (1),

a motorized winch (8) which supports the harness (47) configured to support the patient's weight; infrared light sources (43) directed so to be incident on the rear on the leg muscles at the rear femoral zone, at the rear ankle zone; at the front femoral zone; at front ankle zone; at the glutei zone;

a horizontal pelvic support (12), hinged to a vertical axis and provided with adjustable rigidity springs (31) and an adjustable dampening hydraulic dampener (30);

a motorized exoskeleton for lower limbs (11), configured such that it is fastened to the patient femur, tibia and foot and so that it guides his movements while walking in a combined manner with the movement of the tapis roulant (1);

a user interface (22) in visible position for the user;

a data processing and control unit (10), which manages the frequency of switching on and the intensity of the infrared light sources (43) in a differentiated manner for each light source provided, combining it with the automatized movement of the motorized exoskeleton for lower limbs (11).

It has been said also that the action of infrared radiation combined with the movement of the exoskeleton is both to heat the muscle lighted by the source and to provide the user a proprioceptive stimulation, since the switching on the source combined with the single movement can be perceived by the user who combines it then sensorially with the movement.

Moreover, in contact to the legs and/or arms muscles a plurality of electrodes can be arranged, which are controlled by the data processing and control unit (10), which are configured to stimulate the muscles in a combined manner with the movement induced by the lower and upper limbs orthoses.

In a preferred embodiment, on said processing and control unit (10) computer programs are stored, which are configured to implement the following method:

1) subdividing the lower limbs in a plurality of zones, each zone being associated to the stimulation by means of one of the infrared light sources provided in the device.

Preferably, for each side of the patient (right/left) a zone is associated to the gluteus, a zone is associated to the front femoral part, a zone is associated to the rear femoral part, a zone is associated to the front tibial part, a zone is associated to the rear tibial part.

2) Schematizing, for each one of said zones defined in point 1, the action cycle imposed by said motorized exoskeleton as a sequence of a plurality of contraction and relaxation steps of the main muscular groups provided in each zone; (3) determining, for each light source, an intensity adjusting cycle as a function of the contraction and relaxation cycle schematized in point 2) for the zone associated to the specific light source.

The intensity adjusting cycle is preferably configured such that each light source is switched on at the contraction step of the relative muscle group; in an embodiment, each light source can be switched on at a defined time before the contraction step, so to provide a stimulation able to “inform” the user of the need to contract the relative muscle group.

Preferably, but not limitingly, the method comprises also the steps of:

4) determining, for each electrode configured to stimulate the muscle groups present in one of the zones defined in point 1), a muscular stimulation cycle as a function of the contraction and relaxation cycle, schematized in point 2).

The stimulation cycle is preferably configured such that each electrode stimulates the muscles at which it is installed during the contraction steps of the relative muscular group.

In an embodiment, each electrode can be actuated at a defined time before the contraction step, so to provide a stimulation able to “inform” the user of the need to contract the relative muscular group.

In another embodiment, the device is characterized in that said exoskeleton is configured to measure the muscular effort exerted by each muscular group associated to each one of said zones defined in point 1, and in that the intensity adjusting cycle is configured such that each one of said light sources (43) can be adjusted so that its intensity varies proportionally to the intensity of the effort exerted by the muscular group lighted.

This synchrony between thermal—and possibly electrical—stimulations and the walking cycle amplifies the user perception of his movement sensibly, and so it speeds up the rehabilitation cycle.

In another embodiment, the device comprises also an exoskeleton for upper limbs, configured to guide the movement of the arms of the user in a coordinated manner with the movement of said exoskeleton for lower limbs. Coordinated manner means that an oscillatory movement is imposed to the arms of the user, typical of the physiological walking. In this embodiment, the device comprises also a plurality of further light sources configured to light each of the muscles of a zone of upper limbs of the patient and is characterized in that said processing unit is configured to manage an intensity adjusting cycle of said further light sources in a differentiated manner for each one of said sources, as a function of the movement cycle imposed by said exoskeleton to the upper limbs.

1. A rehabilitation device, comprising:
a motorized treadmill (1),
a harness (47) configured to support the weight of the patient;
a plurality of infrared light sources (43), each one directed to light the muscles of a zone of the lower limbs of the patient,
a motorized exoskeleton for lower limbs (11), configured such that it is fastened to the patient femur, tibia and foot and such that it guides patient movements while walking in a combined manner with the movement of said motorized treadmill (1),
a user interface (22), arranged in a position visible for the user;
a data processing and control unit (10), configured to control said motorized exoskeleton (11) and said light sources (43),
and wherein
said processing and control unit (10), is configured to manage an intensity adjusting cycle of said light sources (43) in a differentiated manner for each one of said sources, as a function of the movement cycle imposed by said exoskeleton (11) to the lower limbs.

2. The device according to claim 1, further comprising, in contact to the legs muscles of the patient, a plurality of electrodes, controlled by said data processing and control unit (10) and configured to stimulate the muscles as a function of the movement cycle imposed by said exoskeleton (11) to the lower limbs.

3. The device according to claim 1, wherein on said processing and control unit (10) computer programs are stored configured to implement the following method:
1) subdividing the lower limbs in a plurality of zones, each zone being associated to the stimulation by means of one of said infrared light sources (43);
2) schematizing, for each one of said zones defined in point 1), the action cycle imposed by said motorized exoskeleton (11) as a sequence of a plurality of contraction and relaxation steps of the main muscular groups provided in each zone;
3) determining, for each one of said light sources, an intensity adjusting cycle as a function of said contraction and relaxation cycle schematized in point 2) for the zone associated to the specific light source.

4. The device according to claim 3, wherein said intensity adjusting cycle is configured such that each light source is switched on at the contraction steps of the relative muscular group and is switched off at the relaxation steps of the relative muscular group.

5. The device according to claim 3, wherein each light source (43) is switched on at a defined time before the contraction step of the relative muscular group.

6. The device according to claim 3, wherein said motorized exoskeleton (11) is configured to measure the muscular effort exerted by each muscular group associated to each one of said zones defined in point 1, and wherein
the intensity adjusting cycle is configured such that each one of said light sources (43) can be adjusted so that its intensity varies proportionally to the intensity of the effort exerted by the muscular group lighted.

7. The device according to any one of claims 3, wherein said zones defined in point 1) comprise, for each side of the patient, a zone associated to the gluteus, a zone associated to the front femoral part, a zone associated to the rear femoral part, a zone associated to the front tibial part, a zone associated to the rear tibial part.

8. The device according to claim 3, wherein said method comprises further the steps of:
4) determining, for each electrode configured to stimulate the muscle groups present in one of the zones defined in point 1),
5) a muscular stimulation cycle as a function of the contraction and relaxation cycle, schematized in point 2).

9. The device according to claim 8, wherein said muscular stimulation cycle is configured such that each electrode stimulates the muscles at which it is installed during the contraction steps.

10. The device according to claim 8, wherein said stimulation cycle is configured such that each electrode stimulates the muscles at which it is installed at a defined time before the relative contraction step.

11. The device according to claim 1, further comprising a horizontal pelvic support (12), hinged to a vertical axis and provided with variable rigidity springs (31) and with an adjustable dampening hydraulic dampener (30).

12. The device according to claim 1, further comprising-also an exoskeleton for upper limbs, configured to guide the movement of the arms of the user in a coordinated manner with the movement of said exoskeleton for lower limbs, and comprising also a plurality of further light sources configured to light each muscle of a zone of the upper limbs of the patient
and wherein said processing and control unit (10), is configured to manage an intensity adjusting cycle of said further light sources in a differentiated manner for each one of said sources, as a function of the movement cycle imposed by said exoskeleton to the upper limbs.
